(19) United States
(12) Patent Application Publication
(10) Pub. No.: US 2006/0271171 A1
(43) Pub. Date: Nov. 30, 2006

(54) Artificial heart valve

(76) Inventors: Tim C. McQuinn, Charleston, SC (US); Richard Figliola, Central, SC (US); Donald Beasley, Seneca, SC (US)

Correspondence Address: NEEDLE & ROSENBERG, P.C., SUITE 1000, 999 PEACHTREE STREET, ATLANTA, GA, 30309-3915, US

(21) Appl. No.: 11/396,947

(22) Filed: Apr. 3, 2006

Related U.S. Application Data
(60) Provisional application No. 60/667,465, filed on Apr. 1, 2005.

(51) Int. Cl. A61F 2/24

(52) U.S. Cl. 623/2.1; 623/2.38

(57) ABSTRACT

Provided herein is a cardiac valve prosthesis comprising a annulus body having a back face and defining a passage for the flow of blood along an axis of the passage. The passage has a first open end having a greater diameter that its opposed second open end. In a further aspect, the valve prosthesis includes means for creating a fluid barriers to the flow of fluid therethrough the passage of the annulus body.

### CROSS-REFERENCE TO RELATED APPLICATIONS

This application claims the benefit of U.S. Provisional Application No. 60/667,465, filed on Apr. 1, 2005, which is incorporated by reference herein in its entirety.

### ACKNOWLEDGEMENTS

This invention was made with government support under Grants EPS-0132573 from the National Science Foundation and 8-PORR16461 from the National Institutes of Health. The government has certain rights in the invention.

### BACKGROUND

The proper functioning of the human heart is vital for survival and maintaining an active lifestyle. Heart valve problems often are severe enough to require a valve replacement. Valve replacement is typically accomplished with mechanical valves or with bioprosthetic or cryopreserved valves. Mechanical valves typically comprise an annular ring, occluder or plate leaflets(s), and retaining strut(s) or hinges. Bioprosthetic valves are heart valves harvested from animals or cadavers that are treated to prevent rejection or rapid degradation.

One of the major issues with valve replacement using mechanical prostheses is thromboembolic complication. Thrombus formations can hinder the motion of a valve's mechanism and cause it to be stenosed, regurgitant, or stuck in an open or closed position. This has been a major problem in mechanical prostheses in the pulmonary position in adults and children. Miyamura, H., et al., (1987) J. Thoracic and Cardiovascular Surg. 94, 1:148-150.

Another complication that can hinder the performance of a mechanical valve is pannus growth. Pannus is tissue overgrowth on a portion of the valve, which can be just as much of a problem as thrombus formation, as it too can clog or stenose a valve. Pannus formation is an especially severe problem in the pulmonary position where it has been found that pannus formation is a primary cause of mechanical valve malfunction. Ilbawi, M., et al., (1987) J. Thoracic and Cardiovascular Surg. 93, 1:73-79.

Failure of the valve mechanism is another potential risk of using a mechanical valve. A mechanical valve is subject to many opening and closing cycles. Mechanical valve failure is a very serious event and usually requires immediate surgery.

Bioprosthetic or cryopreserved valves deteriorate and have a limited lifespan. Cryopreserved and bioprosthetic valves will not last through a young adult or adolescent's lifespan, thus mandating at least one additional operation.

Overall, mechanical valves have a high rate of thrombotic and pannus complications especially when placed in the pulmonary position. Further, cryopreserved and bioprosthetic valves do not offer a long term solution. Thus, suitable prosthetic valve solutions for the pulmonary position are not available.

### SUMMARY

According to one embodiment of the invention, a cardiac valve prosthesis comprises a annulus body defining a passage for the flow of blood from a first open end to a second open end. In one aspect, the first open end can have a greater diameter than the second open end. In a further aspect, the valve prosthesis further comprises means for creating a fluid barrier to the flow of fluid therethrough the passage of the annulus body, such that, in use, the valve prosthesis of the present invention provides a preferred direction of blood flow (i.e., from the first open end toward the second open end), such as during systole, and to offer an increased resistance to blood flow in the opposite retrograde direction, such as during diastole, so as to restrict the volume of blood in the retrograde direction relative to the preferred direction. It is contemplated that the described valves can be used in a cardiac surgery method for replacing a cardiac valve in a subject.

Other apparatus, methods, and aspects and advantages of the invention will be discussed with reference to the Figures and to the detailed description of the preferred embodiments.

### BREIF DESCRIPTION OF THE DRAWINGS

The figures are not necessarily to scale, emphasis instead being placed upon illustrating the principles of the invention. These and other features of the preferred embodiments of the invention will become more apparent in the following detailed description in which reference is made to the appended figures wherein:

FIG. 1 is a front perspective view showing an embodiment of the cardiac valve prosthesis.

FIG. 2 is a rear perspective view of the cardiac valve prosthesis of FIG. 1.

FIG. 3 is a cross sectional view of the cardiac valve prosthesis taken across line 3-3 of FIG. 1.

FIG. 4 is a cross sectional view showing an alternative embodiment of the cardiac valve prosthesis.

FIG. 5 is a front perspective view showing an alternative embodiment of the cardiac valve prosthesis.

FIG. 6 is a rear perspective view of the cardiac valve prosthesis of FIG. 5.

FIG. 7 is a cross sectional view of the cardiac valve prosthesis taken across line 7-7 of FIG. 5.

FIG. 8 is a perspective cross sectional view of the cardiac valve prosthesis taken across lines 7-7 and 8-8 of FIG. 5.

FIG. 9 is a perspective view showing an exemplary conduit or portion thereof.

FIG. 10 is a rear perspective view showing an alternative embodiment of the cardiac valve prosthesis.

FIG. 11 is a cross sectional view showing an alternative embodiment of the cardiac valve prosthesis.

FIG. 12 is a cross sectional view showing an alternative embodiment of the cardiac valve prosthesis.

FIG. 13 is a perspective view showing an alternative embodiment of the cardiac valve prosthesis.

FIG. 14 is a cross sectional view of the cardiac valve prosthesis taken across line 12-12 of FIG. 13.

FIG. 15 is a perspective view showing an alternative embodiment of the cardiac valve prosthesis.

FIG. 16 is a cross sectional view of the cardiac valve prosthesis taken across line 14-14 of FIG. 15.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used in the specification and the appended claims, the singular forms “a,” “an,” and “the” comprise plural referents unless the context clearly dictates otherwise. For example, reference to a component in the singular is intended to comprise a plurality of components. Thus, reference to “a conduit” includes embodiments with one, two, or more such conduits.

Ranges may be expressed herein as from “about” or “approximately” one particular value and/or to “about” or “approximately” another particular value. When such a range is expressed, another embodiment comprises from the one particular value and/or to the other particular value. Similarly, when values are expressed as approximations, by use of the antecedent “about,” it will be understood that the particular value forms another embodiment.

As used herein, the terms “optional” or “optionally” mean that the subsequently described event or circumstance may or may not occur, and that the description includes instances where said event or circumstance occurs and instances where it does not.

As used in the specification and the appended claims, by a “subject” is meant an individual. The term does not denote a particular age or sex. In one aspect, the subject is a mammal such as a primate, including a human. The term includes human and veterinary subjects.

As used in the specification and the appended claims “systolic” or “forward” blood flow refers to blood flow from the ventricle to the aorta or to the pulmonary artery during contraction of a subject's cardiac ventricle. “Diastolic,” “reverse,” “regurgitant,” “retrograde,” or “backward” blood flow refers to blood flow from the aorta or pulmonary artery back into the ventricle during relaxation of a subject's cardiac ventricle. “Reverse,” “regurgitant,” “retrograde” or “backward” flow can also refer to flow from the ventricle into the atrium across the mitral or tricuspid valve during ventricular systole.

The present invention may be understood more readily by reference to the following detailed description of preferred embodiments of the invention and the examples included therein and to the Figures and their previous and following description.

Provided herein is a cardiac valve prosthesis comprising an annulus body defining a passage therein for the flow of blood therethrough the passage. The passage has a first open end and an opposed second open end. In one aspect, the diameter of the first open end is greater than the diameter of the second open end. In another aspect, at least a portion of an inner surface of the passage tapers inwardly toward the longitudinal axis of the annulus body as the passage extends from the first open end to the second open end. In this aspect, the diameter of the passage in the inwardly tapering portion of the passage decreases as the passage extends toward the second open end.

Optionally, the annulus body further comprises a back face that circumferentially surrounds at least a portion of the second open end of the passage. In one exemplified aspect, the back face extends circumferentially about the second open end of the passage. In a further aspect, the annulus body further defines a first conduit in fluid communication with the back face and the inner surface of the passage. In one aspect, the conduit has a first orifice least partially positioned on the back face and a second orifice positioned on the inner surface of the passage.

In operation, blood flowing during diastole passes into the first conduit and exits out into the passage to act as a fluid barrier to restrict diastolic blood flow reversal through the passage and into a subject's cardiac ventricle. Thus, the blood flowing through a conduit during diastole can reduce regurgitant flow into the subject's ventricle without an occluder, plate leaflet, retraining strut, or hinge that are conventionally used to reduce regurgitant in mechanical prosthetic valves.

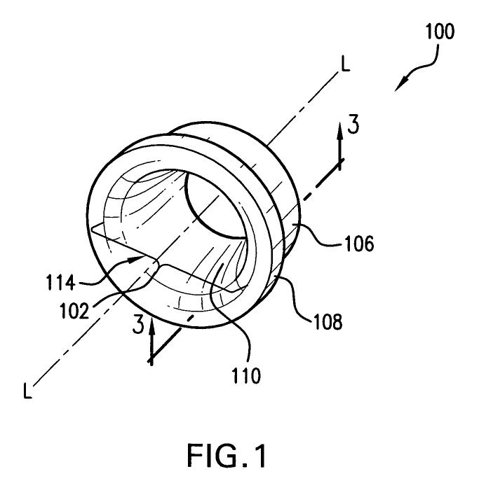

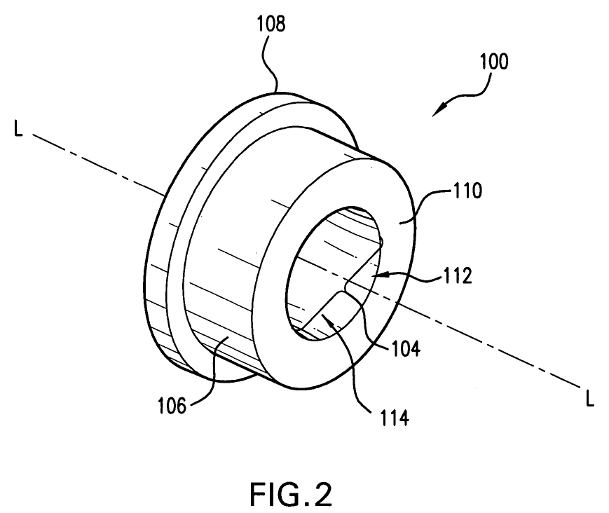

FIG. 1 is a perspective view showing an exemplary cardiac valve prosthesis 100. The cardiac valve prosthesis 100, referred to throughout as “valve,” “prosthesis,” or “cardiac valve prosthesis,” comprises an annulus body 106. As noted above, the annulus body 106 defines a passage 114, which extends through the body along the annulus body's or passage's longitudinal axis (L). The passage has an inner surface 110. The passage further defines a first open end 102 and a second open end 104 (FIG. 2) in the annulus body.

FIG. 2 is a perspective view of the valve 100 showing that the annulus body 106 can further comprise a back face 112 circumferentially surrounding the second open end 104. The back face 112 is located on the systolic outflow end of the prosthesis. By “systolic outflow end” is meant the end of the prosthesis upon which the second open end 104 is defined or the end of the prosthesis where blood flow primarily exits the valve during systole. After blood flows through the valve's passage 114 during systole, it can contact the back face. Generally, blood moving in a backward or retrograde direction during diastole can contact the back face 112. In one aspect, the back face of the prosthesis can be flat. As one will appreciate, it is contemplated that other shapes, such as, for example, concave, convex, sloping and hook shapes, and the like, can be used for the back face.

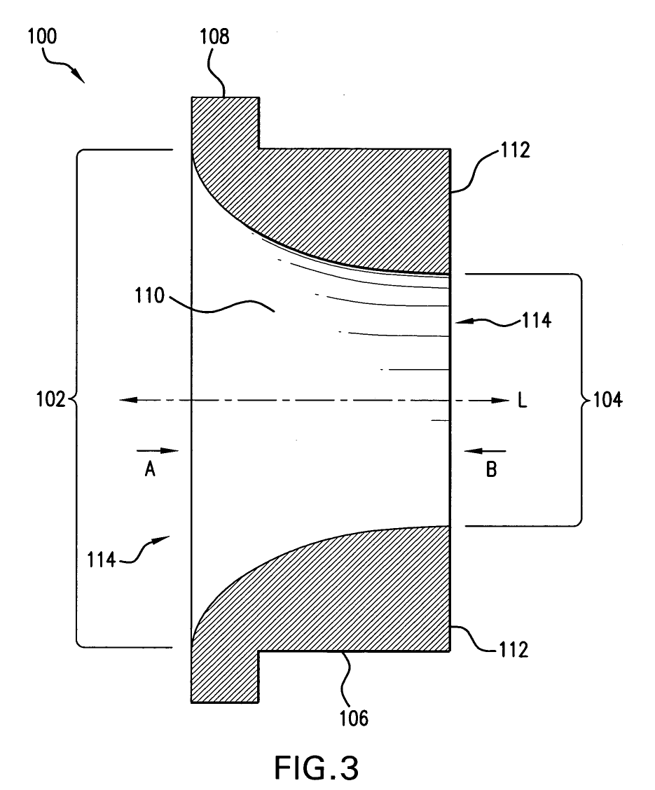

FIG. 3 is a cross sectional view of the valve 100 taken across the line 3-3 of FIG. 1 showing that the passage 114 allows for blood flow along the axis (L) of the passage in the forward or systolic direction shown by arrow A and in a reverse or diastolic direction shown by arrow B.

Referring to FIGS. 1-3, the valve's passage 114 has a first open end 102 of a first diameter and a second open end 104 of a second diameter. The diameter of the first open end 102 is larger than the diameter of the second open end 104. At least a portion of the passage tapers or contracts in size as it extends from the first open end 102 to the second open end 104.

Thus, the passage 114 tapers or contracts in the direction of forward or systolic blood flow. During systole, blood enters the passage 114 through the first open end 102 and exits the passage through the second open end 104. During diastole, blood can flow in a reverse direction by entering the second open end 104 and flowing through the passage 114 towards the first open end 102.

It is contemplated that the passage 114 can taper or contract smoothly from the first open end 102 to the second open end 104. In one example, such a smooth contraction can be based on an elliptical surface, meaning that the passage contracts with a substantially elliptical curvature. The passage of the exemplary valve 100 can contract with a substantially elliptical curvature.

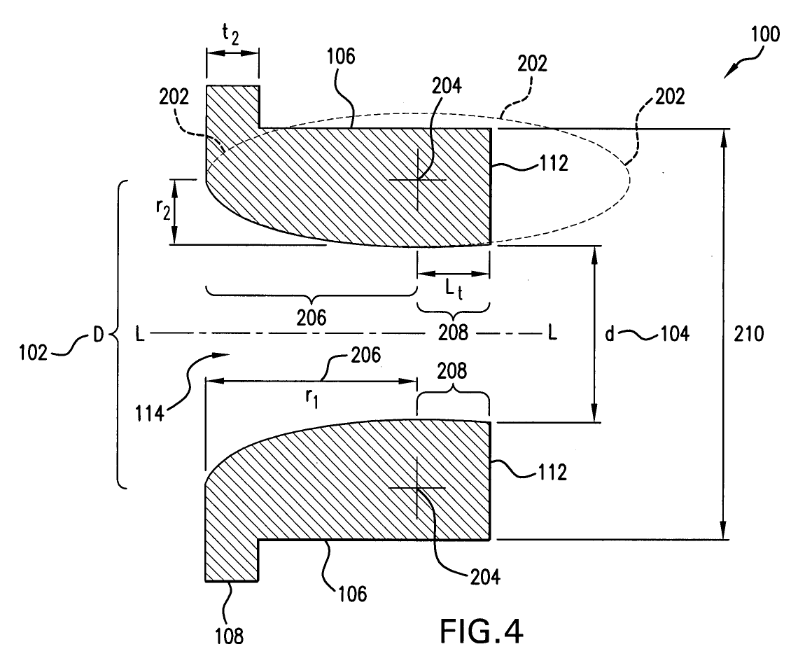

FIG. 4 is another cross sectional view of the valve 100 across line 3-3 of FIG. 1, demonstrating a substantially elliptical contraction or taper. As illustrated, the taper of the passage 114 can be based on an ellipse 202. The centroid 204 of the ellipse can be located within the annulus body 106 of the valve, as shown in FIG. 4, or outside of the annulus body (not shown). The centroid may be located within or outside of the annulus body of the valve by varying the length of Lₜ. For example, by lengthening Lₜ, the centroid may be within the annulus body of the prosthetic valve or by shortening Lₜ, the centroid of the ellipse may be located outside of the annulus body of the prosthesis.

An elliptically shaped contraction, however, is only one example of a smooth contraction that the passage 114 can take. Optionally, the passage's contraction can be shorter or longer than the contraction defined by an ellipse. For example, and not meant to be limiting, the contraction can be defined by a circular shape, by a cubic polynomial described by Y=X³, or one based on splines. In general, the passage's contraction can be defined by any elliptical, circular or polynomial shape or collocation method, or otherwise, that determines a contour from the first open end 102 to the second open end 104. Other curved shapes that the passage's contraction can include, but are not limited to, are curves generated by co-localization points and curve-fits, such as splines. Any contracting geometric shape can be used that defines a taper of the passage from the first open end 102 to the second open end 104.

When shapes are used to define a more rapid contraction, for example a circle, the valve prosthesis may have an elongated Lₜ portion. Thus, a inwardly tapering or contracting portion 206 (r₁) of the passage starting from the first open end 102, can be followed by a second passage portion. In varying aspects, the second passage portion can comprise a consistent, or non-contracting portion 208 (Lₜ) of the passage. In this aspect, the second passage portion extends substantially parallel to the longitudinal axis of the annulus body. Alternatively, the second passage portion can comprise an outwardly tapering or expanding portion. In this aspect, the second passage portion could be curved in shape. In a further aspect, the second passage portion 208 can vary in length depending on the geometric shape of contraction and the overall length of the prosthesis. Valves with a longer Lₜ section generally have their centroid located in the annulus body of the valve. As Lₜ shortens, and eventually moves to 0, the centroid of the ellipse moves towards the back face 112 of the valve. In is contemplated, in an alternative aspect, that the centroid can be located outside of the annulus body 106 when Lₜ=0 (not shown).

By contracting in the direction of systolic flow, the valve shows a higher pressure drop in the direction of diastolic flow that systolic flow. Pressure drop is a measure of flow resistance. A higher pressure drop in equates to reduced flow under the same conditions. As such, the valve allows a higher blood flow rate in the forward or systolic direction than is allowed in the reverse or diastolic direction.

Not only can the taper of the passage 114 be varied based on different geometric shapes, the diameter (D) of the first open end 102 and the diameter (d) of the second open end 104 can be varied in relation to one another and absolutely. This relationship of large diameter 102 to small diameter 104 can be defined as β, which can be described by the following equation: β=the second open end's 104 diameter (d) divided by the first open end's 102 diameter (D), or β=d/D. The Beta value β can range from between about 0 to 1.0. In some preferred embodiments, the beta value is between about 0.4 to about 0.8. In other preferred embodiments, the beta value is between about 0.5 to about 0.7.

Because the first diameter, D, is generally fixed at the tissue annulus diameter 210 of a subject, which, for example, is about 27 millimeters for adults or about 25 millimeters for adolescents, the beta value (β) is generally varied by changing the diameter of the second open end 104.

The absolute size of the diameters d and D can also be varied together or independently depending on the size of the valve desired for placement into a subject, desired flow characteristics, and depending on which of the subject's valve is replaced. Thus, the absolute diameter of d and D can vary depending on factors including, for example, whether the prosthetic valve is replacing an aortic, pulmonary, mitral, or tricuspid valve, or on the gender, age, size, medical condition, heart size, and valve size of a given subject. The absolute size of d and D therefore should not be considered to limit the scope of the invention. The described beta ratios and geometric taper shapes can be achieved with diameters of any desired absolute size. The absolute length of the valve can also vary. In one example, not meant to be limiting, the length can be D divided by 2 (D/2).

In some embodiments, the beta ratio and the shape of the passage's 114 taper can change in vivo. For example, as the pressure gradient across the valve changes between systole and diastole, the beta ratio and shape of the passage can change. For example, the diameter D of the first open end 102, the diameter d of the second open end 104 or the diameter 210 (FIG. 2) can change during systole or diastole. An increase in the diameter d of the second open end 104, or the diameter of 210 reduce transvalvular pressure allowing for increased systolic flow. A decrease in the diameter d of the second open end 104 or of the diameter 210 causes an increase in diastolic flow resistance during diastole, which reduces reverse flow through the second open end 104. In aspects where the beta ratio and the shape of the passage's 114 taper can change in vivo, at least a portion of the valve is formed of flexible material such that at least a portion of the valve is moveable between a first position having a first diameter and a second position having a second diameter that is less than the first diameter.

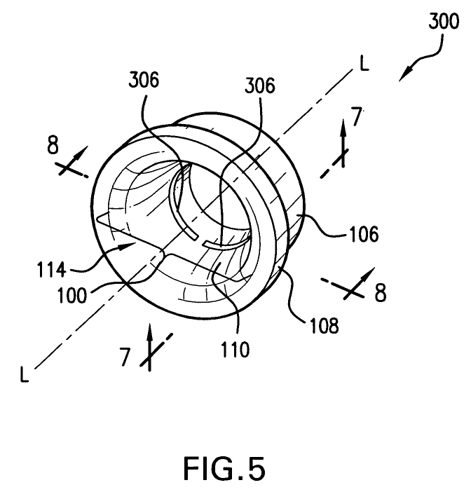

FIG. 5 is a perspective view showing an exemplary prosthetic valve 300 with backflow channels or conduits 302. The valve 300 can be understood as a modification of the valve 100 described above and depicted in FIGS. 1-4. Similar to the valve described above, the valve 300 comprises an annulus body 106 defining a passage 114 for the flow of blood along a longitudinal axis (L) of the passage. As described above, the diameter of the first open end 102 is greater than the diameter of the second open end 104 and the passage tapers in size as it extends from the first open end to the second open end. Moreover, all of the parameters of the passage taper, the ratio of diameter of the first to the second open end, the absolute size of the valve, placement sites in a subject, and other details of the valve and method of use descried above, can apply to the valve 300.

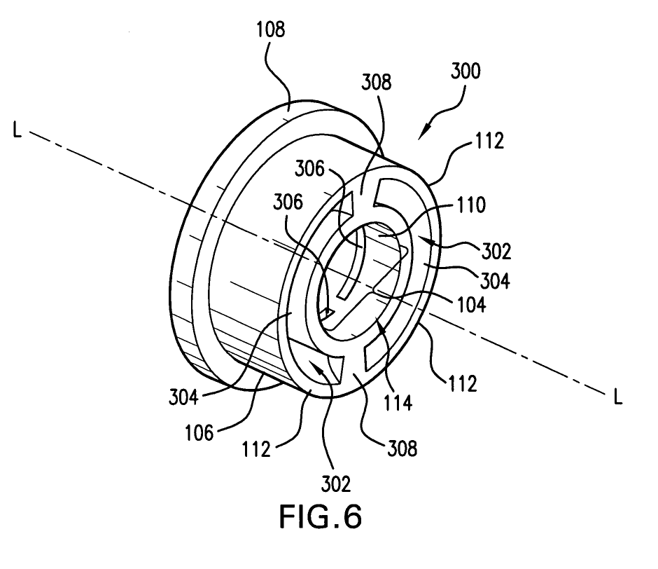

FIG. 6 is a perspective view showing that the valve 300 further comprises a back face 112 circumferentially surrounding the second open end 104, and the annulus body 106 further defines a first conduit 302 for the flow of blood that is in fluid communication with the back face and the inner passage of the annulus body. In this aspect, the first conduit has a first orifice 304 defined at least partially on the back face 112 of the annulus body and a second orifice 306 defined on the inner surface 110 of the passage.

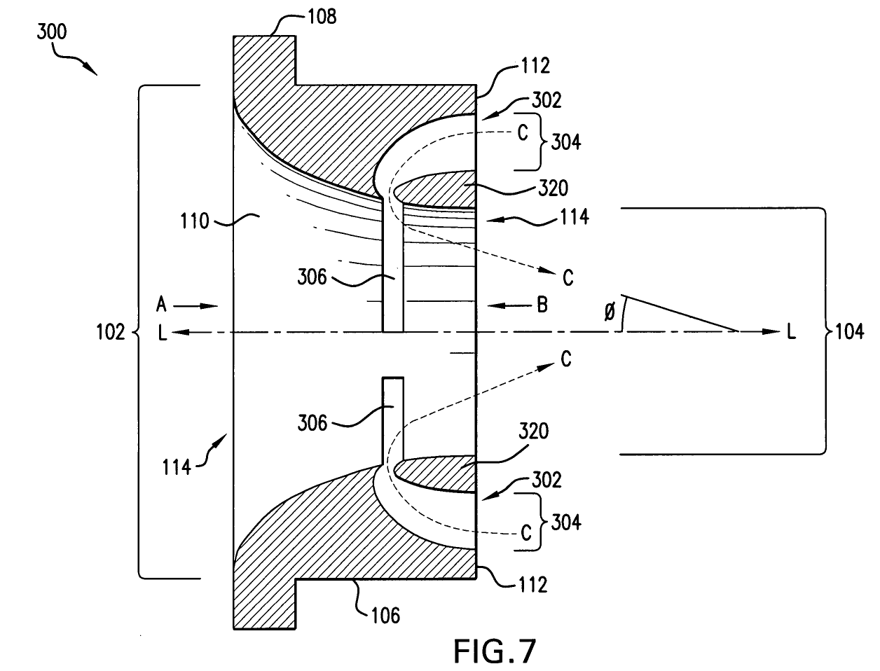

FIG. 7 is a cross sectional view of the valve 300 across the line 7-7 shown in FIG. 5. Referring to FIG. 7, blood can enter the first orifice of the first conduit 302 during diastole in the retrograde direction shown by arrow C. During diastole, blood can flow into the valve passage 114 through the second open end 104 and concurrently also flows into the first orifice 304 of the first conduit 302. After entering the first orifice 304, blood flows through the first conduit 302 and exits the second orifice 306 into the passage 114. Thus, during diastole, blood may enter the valve's passage 114 through the second open end 104 and through the second orifice 306 of the first conduit.

When blood flows out through the second orifice 306 into the passage 114, the blood flowing out of the second orifice 306 can impinge on or oppose the blood that entered the passage through the second open end 104 and that is flowing through the passage 114 along the axis of the passage (L) in the diastolic direction (B). This impingement of blood flow through the passage during diastole, caused by the fluid barrier formed by the blood flow through the first conduit during diastole, reduces the volume of blood exiting out of the first open end of the cardiac valve in the diastolic direction (B). Reducing the volume of blood backflow through the valve's passage 114 reduces the regurgitant fraction of the valve, which is a measure of the volume of blood that returns to a given heart chamber during diastole relative to the forward flow volume during systole.

The angle at which blood flows out of the first conduit's second orifice 306 and into the passage 114 can be described in relation to a longitudinal axis (L) of the passage along the direction of blood flow. Blood flowing out of the first conduit 302 through the second orifice 306 can enter the passage 114 perpendicular to the longitudinal axis (L) of the passage. The blood can also enter the passage 114 at an acute angle θ relative to the longitudinal axis (L) of the passage in the direction of the second open end 104. The angle θ at which the first conduit 302 directs blood into the passage 114 during diastole can range, relative to the longitudinal axis (L) of the passage, from between about 90 degrees (perpendicular) to about 30 degrees in the direction of the second open end 104.

It will be appreciated that the second orifice 306 can be located on any portion of the inner surface 110 of the passage. In one aspect, the second orifice 306 is located on the inwardly tapering or contracting portion 206 of the valve's passage. In another aspect, the second orifice 306 is located on the second passage portion 208 of the valve's passage.

The area of the first orifice 304, or, in embodiments comprising a plurality of first conduits, the area of the plurality of first orifices 304, can be varied relative to the area of the second orifice 306 or the plurality of orifices. Such a relation can be described by the following equation:
α=area of the first orifice 306(a₁) divided by area of the second orifice 304(a₂),
or, α=a₁/a₂.

Generally, this ratio ranges from about 1.0 to about 0.8. Moreover, the absolute area of the back face taken up by the first orifice 304, or plurality thereof, can range from between about 20% to about 100% of the total area of the back face without any orifice.

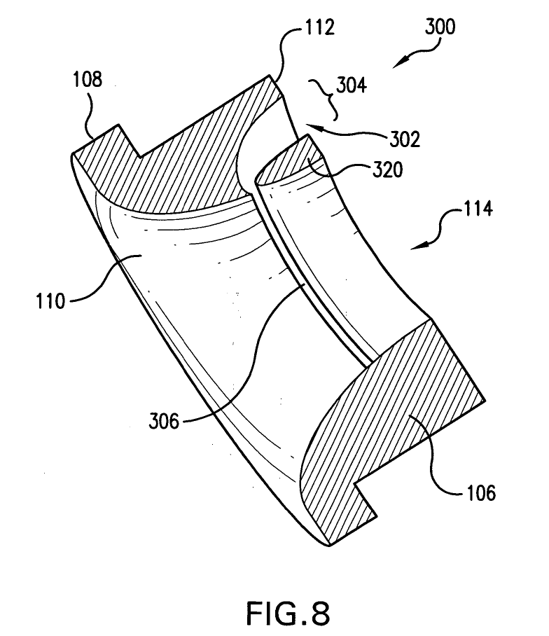

FIG. 8 is a partial perspective view of the valve 300 showing wherein the α ratio is less than about 1.0. The α ratio can be varied by changing the area of either a₁ or a₂. In this illustrated aspect, the first conduit 302 is shown angling back acutely relative to the longitudinal axis of the passage (L), such that blood flowing through the conduit can enter the passage at the desired angle θ.

As shown in the figures, a plurality of first conduits can be used. In one embodiment, two conduits are used. In one aspect, the first orifice 304 of a first conduit can be partially annular in shape. The first orifice can be formed in the back face 112 and can partially surround the second open end 104. In one embodiment, two conduits can be used, wherein each first conduit has a first orifice 304 that partially annular in shape and wherein the first orifice 304 of one of the conduits and the first orifice of the second conduit 304 is separated by a portion 308 of the back face 112. Optionally, each partially annular orifice can sweep across approximately 150 degrees of the back face 112 and be separated by approximately 30 degrees of the back face portion 308. In one aspect, the first orifice 304 can be completely annular in shape and can surround the second open end 104 on the back face 112. In this aspect, only one first orifice 304 is used and there are no separating portions 308 of back face. When the first orifice 304 is completely annular at least a portion (not shown) of the valve annulus body 106 can form a support to maintain the conduit's 302 shape.

In one aspect, the first conduit 302 is typically defined by the annulus body 106 such that a portion 320 of the annulus body 106 separates blood flow through the conduit 302 from blood flowing through the passage 114. The separation of blood flow provided by the portion 320 can be achieved using a separating member (not shown), which can be similar in shape to the annulus body portion 320. The separating member can comprise any suitable biocompatible material. For example, the separating member can comprise at least one of biocompatible materials listed below. Moreover, the separating member can comprise the same or different materials than the valve annulus body 106. In embodiments where a separating member is used, the separating member can be supported in place by vanes, struts, pins, or other structural features as would be clear to one skilled in the art, or combinations thereof. In one aspect, at least one structural feature can extend from the valve annulus body 106 and connect to the separating member.

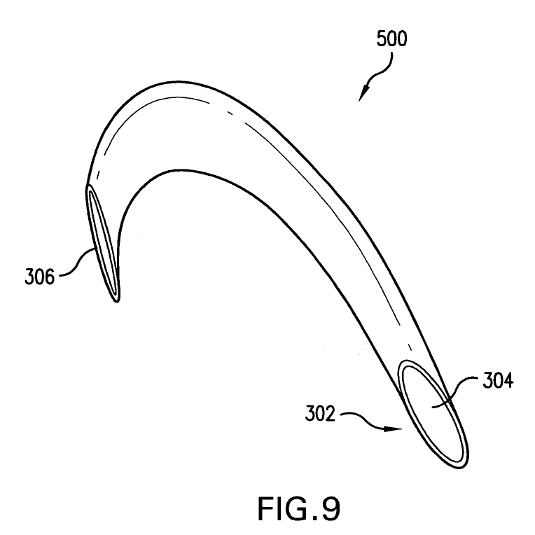

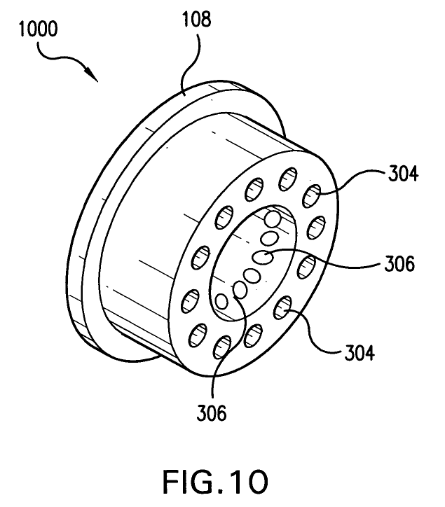

One will appreciate that the shape of the first orifice is not limited to annular or partially annular shapes. For example, as shown in FIG. 10, which is a perspective view of an exemplary valve 1000, a first conduit can have a generally tubular shape with a substantially circular first orifice 308 and a substantially circular second orifice. In general, the shape of the first conduit 302 is related to the shape of its first orifice 304 and second orifice 306. For example, if the first orifice is substantially circular 304 then the shape of the conduit 302 is substantially tubular, or circular in cross section, and the shape of the second orifice 306 is substantially circular. Similarly, if the shape of the first orifice 304 is annular or partially annular, then the shape of the first conduit 302 is typically has an annular cross section, and the second orifice 306 is annular or partially annular in shape. Such a relationship is not, however, required, and any combination of shape of a conduit, first orifice, and second orifice, can be used. In a further aspect, a first orifice, whether annular, partially annular or otherwise shaped, can sweep across from about 1 degree of the blackface to about 360 degrees across the back face. FIG. 9 is a perspective view showing a profile or partial profile 500 of an exemplary conduit 302.

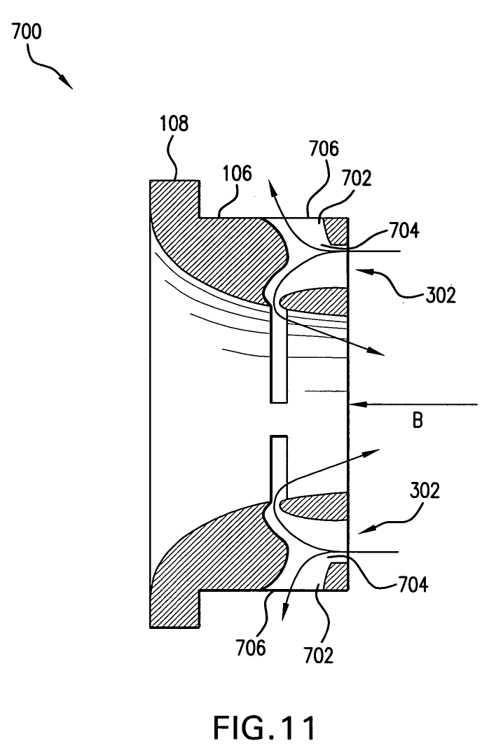

The described valve can be used to replace any cardiac valve of a subject. For example the described valve can be used to replace a subject's pulmonary, aortic, mitral, or tricuspid valve. FIG. 11 shows a cross sectional view of an exemplary valve 700 for placement in the aortic valve position of a subject. The valve 700 can define at least one second conduit 702 in annulus body that is in fluid communication with the first conduit such that blood can be directed to the coronary ostia. In this aspect, the second conduit 702 defines a first open end 704 on the inner surface of the conduit 302 and a second open end 706 on the outer surface of the annulus body 106.

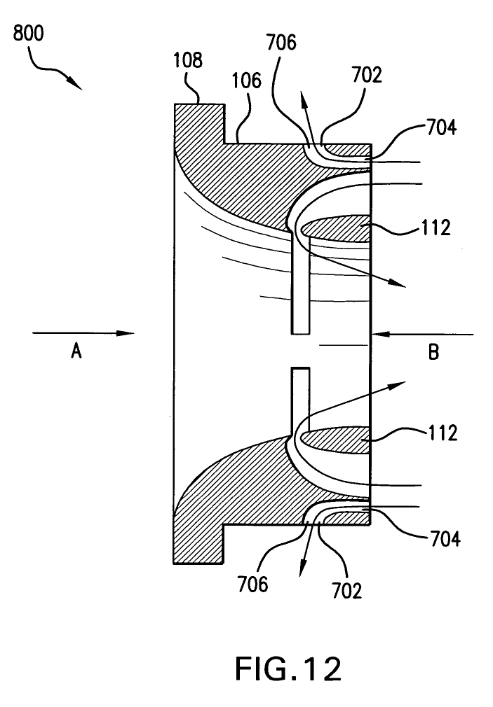

Alternatively, as shown in FIG. 12, a valve 800 designed for replacement of the aortic valve can comprise a second conduit 702 that defines a first open end 704 located on the back face 112 of the annulus body and a second open end 706 located on the outer surface of the valve's annulus body 106. As one will appreciate, in use, diastolic blood flows through the duct 708 and into the coronary ostia.

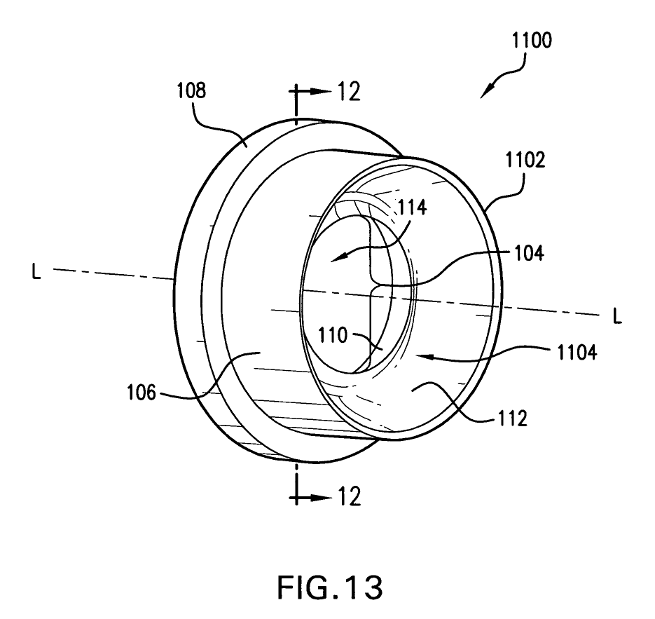

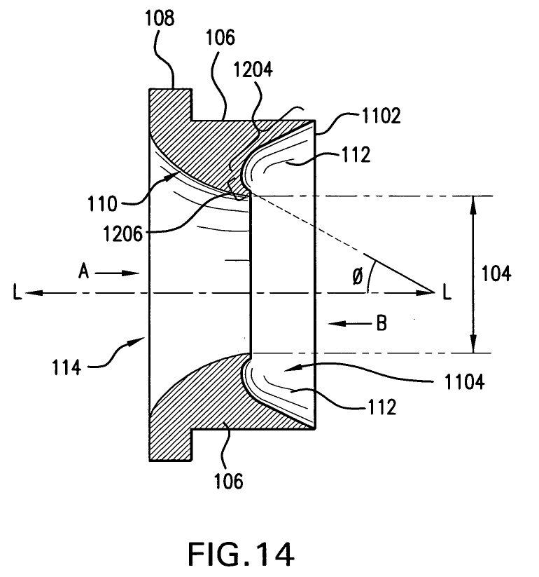

Referring now to FIG. 13-16, an exemplary prosthetic valve 1100 is illustrated. The backface 112 of valve 1100 defines a circumferentially extending trough 1104 that has a substantially hook-shaped cross-sectional shape from the systolic outflow end 1102 towards the second open end 104. In this aspect, the second open end 104 is defined by the junction of the trough 112 and the inner surface 110 of the passage 114. In one aspect, the trough 1104 comprises a first sloping portion 1204 that slopes away from the systolic outflow end 1102 and towards the longitudinal axis (L) of the passage and a second sloped portion 1206 that slopes towards the systolic outflow end 1102 and towards the longitudinal axis (L) of the passage. In one aspect, the first sloped portion 1204 and the second sloped portion 1206 of the trough 1104 form the substantially hook-shaped cross-sectional shape of the trough 1104. In another aspect, at the junction of the trough and the inner surface 110 of the passage 114, the slope of the back face 112 is directed towards the longitudinal axis (L) of the passage and towards the systolic outflow end 1202.

During diastole, blood flows along the trough and is directed into the diastolic flow of blood to impinge on or oppose the blood flowing through the valve along the longitudinal axis (L) of the passage in the diastolic direction (B). This fluid barrier that impinges or blocks at least a portion of the blood flow through the valve during diastole, caused by blood flow directed by the sloped back face 112, reduces the volume of blood flowing through the passage 114 of the valve in the diastolic direction (B). Reducing the volume of blood that backflows through the valve's passage 114 reduces the regurgitant fraction of the valve, which is a measure of the volume of blood that returns to a given heart chamber during diastole relative to the forward flow volume during systole.

The angle at which blood flows off of the juncture of the trough and the second open end of the valve into the primary flow of blood during diastole can be described in relation to a longitudinal axis (L) of the passage. Blood exiting the trough 1104 enters the diastolic flow of blood at an acute angle θ relative to the longitudinal axis (L) of the passage in the direction of the systolic outflow end 1102. The angle θ at which the sloped back face 112 directs blood into the diastolic flow can range, relative to the longitudinal axis (L) of the passage, from between about 90 degrees (perpendicular) to about 40 degrees in the direction of the second open end 104.

The overall size of a cardiac valve prosthesis can be varied depending on the desired size for use in a given subject. For example, younger subjects may require smaller size valves than mature subjects. Moreover the size of the valve can vary depending on which cardiac valve it is designed to replace and on the medical condition of the subject. The appropriate valve size for a given subject is typically a surgical or medical consideration that can be made by an attending surgeon or by another skilled in the art of cardiac valve replacement.

In one aspect of the present invention, the valve prosthesis is configured such that the passage remains substantially patent during systole and diastole. In a further aspect of the invention, it is contemplated that at least a portion of the annulus body is sufficiently resilient to allow in situ expansion and contraction throughout the cardiac cycle. In another aspect, the annulus body is resilient and is configured such that at least a portion of the passage can narrow during diastole.

In another aspect of the present invention, the annulus body of the cardiac valve is configured to the collapsible for transluminal delivery. In this aspect, the annulus body is configured to be expandable to contact the anatomical annulus of the native valve when the valve prosthesis is positioned in situ. Further, it is contemplated that such an “expandable” valve can be self-expanding. In this aspect, it is contemplated that the annulus body can comprise wire. In this aspect the wire can be formed into an expandable wire mesh.

A cardiac valve prosthesis of the present invention can be secured within a subject using methods known in the art for securing prosthetic cardiac valves. For example, a valve can be placed in a subject in an open heart surgical procedure or another standard surgical approach using surgical and medical methods known in the art. The valve could be manufactured from a rigid or flexible material, such as a metal, ceramic or polymer but not exclusively limited to such materials. Moreover, the valve can be made so that it could be delivered and fixed in place in the pulmonary artery by catheterization techniques known in the art. Such a valve would be designed of compressible materials, such as nitinol or other forms of wire mesh, with or without another material within the mesh or lining the luminal surface of the implanted device.

It is contemplated that the valve prosthesis of the present invention can be deployed by catheterization into a stent placed in the pulmonary artery or directly into the pulmonary artery. Deployment involves collapsing or compressing the annulus body of the valve into the distally positioned sheath of a delivery catheter. The tip of the catheter is advanced into the patient's body to the desired anatomical location using methods know in the art. The desired anatomical position may be the location of the deficient native valve. The cardiac valve is expelled from the tip of the catheter using techniques and materials known in the art. On exiting the catheter tip, valve deploys in the desired location. In one example, the materials of the expandable mesh (nitinol or similar material with the ability to regain its original shape following compression) expand spontaneously to an original shape and size configured to perform its valve function while adhering to the wall of the artery and remaining fixed in place. In another aspect, the valve can be expanded into position at the desired location via the actuation of a balloon operatively connected to the tip of the catheter. The catheter is withdrawn to leave the valve prosthesis to function in place of the deficient native valve. In another aspect, the method may also comprise excising the native valve prior to deploying the valve prosthesis.

In another aspect, meshwork metal stents can be deployed into the pulmonary artery and expanded to the degree that they adhere to the wall of the vessel without migration. In operation, a stent having internal mounts or protrusions that are configured to anchor the annulus body of the valve, is deployed into the desired anatomical location for the valve. Subsequently, the valve is deployed into the stent in such a way that the internal protrusions of the stent prevent migration of the valve. Compressible and non-compressible embodiments can be implanted using standard surgical approaches to valve replacement.

The cardiac valve of the present can further comprise an anchor for engaging the lumen wall. The anchor serves to prevent the migration of the valve prosthesis after placement in the desired anatomical position. In one aspect, the anchor comprises at least one circumferentially extending ring member 108 that is configured for suturing such that the valve can be secured to the luminal wall of the pulmonary, aortic, mitral, or tricuspid of the subject. The ring member 108 can be located on the end of the valve nearest the first open end 102. Additionally, more than one ring member can be used to secure the valve within a subject. For example, a second ring member can be placed at the end of the prosthesis nearest the second open end 104.

The choice of what method to use for securing the valve and whether to use a standard surgical or catheter based placement procedure within a subject are decisions within the skill in the art, and may depend on factors that can be determined by a subject's surgeon. For example, a surgeon may choose an approach for implantation and a method for securing the valve depending on; which valve of the subject is being replaced; on characteristics of the subject's medical or physiologic condition; on the procedure to be performed; or depending on the preferences or skill level of the surgeon.

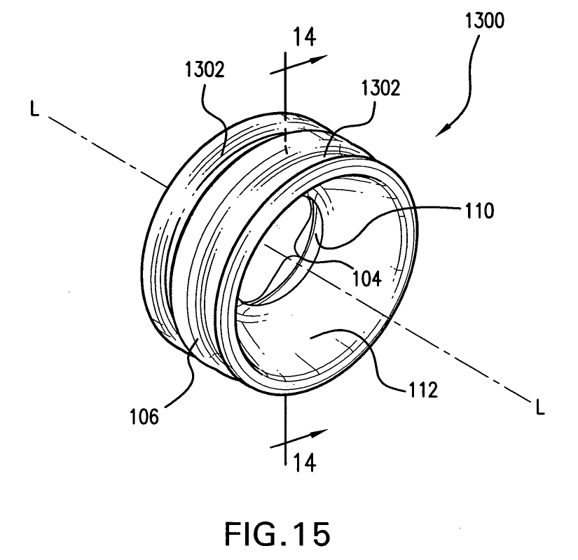

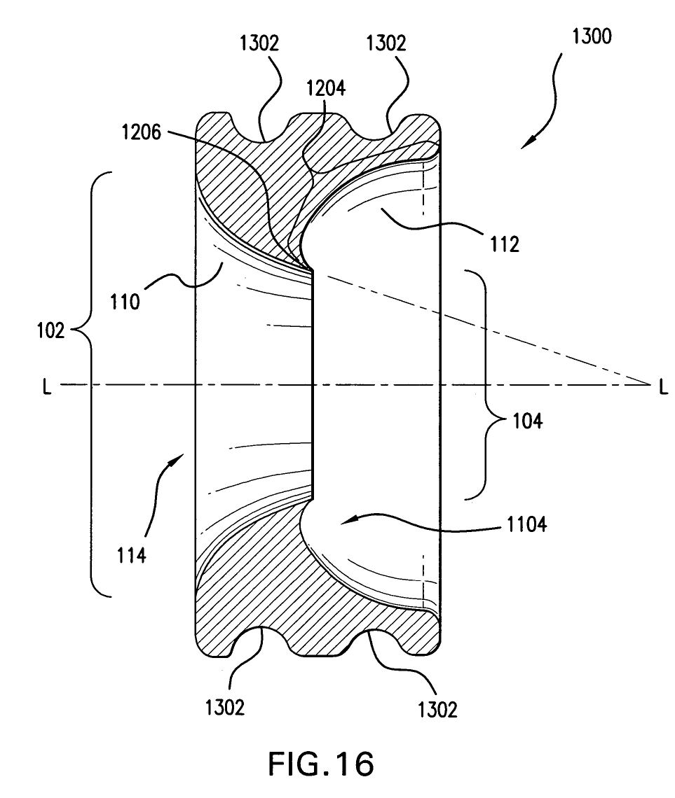

FIGS. 15 and 16 are perspective and cross sectional views of an exemplary valve 1300 that illustrates an alternative attachment strategy. In this aspect, One or more circumferential grooves (1302) that extend about the exterior or outer surface of the annulus body can be used to attach the valve to the vessel using methods known to the art. For example, the grooves can be used to attach one or more separate sewing rings to the valve annulus body or to attach other types of attachment devices used to secure the valve within the vessel.

The cardiac valve prosthesis can be made from any relevant biocompatible material. As used herein, the term “biocompatible material” is used generally to encompass materials that allow interaction with a subject without producing a toxic or injurious response in that subject.

Biocompatible material suitable for valve prosthesis in general include without limitation natural polymers, natural tissues such as treated bovine pericardium, host tissues such as stem cell derived tissues or host pericardium (treated or untreated), synthetic polymers, ceramics, alumina, carbons, turbostratic carbons, pyrolytic carbon, alloyed pyrolytic carbon, silicon-alloyed pyrolytic carbon silicon carbide, graphite, metallics and combinations thereof. Suitable metallic materials include, but are not limited to, metals and alloys based on titanium (such as nitinol, nickel titanium alloys, thermo-memory alloy materials), stainless steel, tantalum, nickel-chrome or certain cobalt alloys including cobalt-chromium-nickel alloys such as Elgiloy and Phynox. Metallic materials also include clad composite filaments, such as those disclosed in WO 94/16646. Suitable biomaterials also include, but are not limited to, coatings of pyrolytic carbon on a substrate of carbon material, ceramic, or metal. The polymer(s) useful for forming the valve prosthesis should be ones that are biocompatible and avoid irritation to body tissue. Suitable polymeric materials include but are not limited to, polyurethane and its copolymers, silicone and its copolymers ethylene vinyl-acetate, polyethylene terephtalate, thermoplastic elastomers, polyvinyl chloride, polyolefins, cellulosics, polyamides, polyesters, polysulfones polytetrafluorethylenes, polycarbonates, acrylonitrile butadiene styrene copolymers acrylics, polylactic acid, polyglycolic acid, polycaprolactone, polylactic acid- polyethylene oxide copolymers, cellulose, collagens, treated bovine pericardium, host tissues such as host pericardium (treated or untreated), stem cell derived tissues, or and chitins.

Other polymers that are useful as materials for valve prostheses include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co- i polymer, polylactic acid, poly(-caprolactone), poly(8-hydroxybutyrate), polydioxanone, poly(-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross linking groups, e.g., Arg-Gly-Asp (RGD), in which the polymers retain their structural integrity while allowing for attachment of molecules, such as proteins, nucleic acids, and the like.

The polymers may be dried to increase its mechanical strength. The polymers may then be used as the base material to form a whole or part of the valve prosthesis.

Furthermore, although the invention can be practiced by using a single type of polymer to form the valve prosthesis, various combinations of polymers can be employed. The appropriate mixture of polymers can be coordinated to produce desired effects when incorporated into a valve prosthesis.

A valve prosthesis of the invention can be coated or impregnated with a therapeutic agent. The term “therapeutic agent” as used throughout encompasses drugs, genetic materials, and biological materials and can be used interchangeably with “biologically active material,” or “pharmaceutical compositions.” Non-limiting examples of suitable therapeutic agents include heparin, heparin derivatives, urokinase, dextrophenylalanine, praline, arginine, chloromethylketone (PPack), enoxaprin, angiopeptin, hirudin, acetylsalicylic acid,; tacrolimus, everolimus, rapamycin (sirolimus), amlodipine, doxazosin, glucocorticoids, betamethas one, dexamethas one, prednisolone, corticosterone, budesonide, sulfas alazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe- Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional; molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17 beta estradiol, digoxin, beta blockers, captopril, enalopril, stating, steroids, vitamins, taxol, paclitaxel, 2′-succinyl-taxol, 2′-succinyl-taxol triethanolamine, 2′-glutaryl-taxol, 2′-glutaryl taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. Optionally, the therapeutic agent is a smooth muscle cell inhibitor or antibiotic. In one aspect, the therapeutic agent is taxol (e.g., Taxol), or its analogs or derivatives. In another aspect, the therapeutic agent is paclitaxel, or its analogs or derivatives. In yet another aspect, the therapeutic agent is an antibiotic such as erythromycin, amphotericin, rapamycin, adriamycin, etc. The term “genetic materials” means DNA or RNA, including, without limitation, of DNA/RNA encoding a useful protein, intended to be inserted into a human body including viral vectors and non-viral vectors.

In one aspect, the pharmaceutical compositions impregnated in or coated on the valve prosthesis are useful for inhibiting the proliferation and/or migration of vascular smooth muscle cell, tumor cell, stromal cell, interstitial matrix surrounding vascular smooth muscle cell or immune system effecter cell. Optionally, the pharmaceutical compositions and valve prosthesis are capable of preventing or treating a proliferative disease, such as restenosis or setnosis.

The pharmaceutical compositions impregnated in or coated on the valve prosthesis may be used to inhibit the proliferation and/or migration of cells of a body tissue, e.g., epithelial tissue, connective tissue, muscle tissue, and nerve tissue. Epithelial tissue covers or lines all body surfaces inside or outside the body. Examples of epithelial tissue include, but are not limited to, the skin, epithelium, dermis, and the mucosa and serosa that line the body cavity and internal organs, such as the heart, lung, liver, kidney, intestines, bladder, uterine, etc. Connective tissue is the most abundant and widely distributed of all tissues. Examples of connective tissue include, but are not limited to, vascular tissue (e.g., arteries, veins, capillaries), blood (e.g., red blood cells, platelets, white blood cells), lymph, fat, fibers, cartilage, ligaments, tendon, bone, teeth, omentum, peritoneum, mesentery, meniscus, conjunctive, aura mater, umbilical cord, etc. Muscle tissue accounts for nearly one-third of the total body weight and consists of three distinct subtypes: striated (skeletal) muscle, smooth (visceral) muscle, and cardiac muscle. Examples of muscle tissue include, but are not limited to, myocardium (heart muscle), skeletal, intestinal wall, etc.

In one aspect, the pharmaceutical compositions impregnated in or coated on the valve prosthesis are capable of inhibiting at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or about 100% (completely) of cell proliferation and/or migration in the cells that were exposed to the therapeutic agent, optionally paclitaxel.

In another aspect, the pharmaceutical compositions impregnated in or coated on the valve prosthesis are capable of reducing at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or about 100% (completely) of the symptoms/severity/degree of restenosis or stenosis.

In other aspects, the valve prosthesis elutes an amount of the therapeutic agent(s) that is capable of inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. Optionally, the amount eluted is capable of altering the cellular metabolism and/or inhibiting cell proliferation and/or migration. Optionally, the cell is a vascular smooth muscle cell, tumor cell, stromal cell, interstitial matrix surrounding vascular smooth muscle cell or immune system effecter cell. Optionally, the amount eluted allows for cellular repair and matrix production. Optionally, the amount eluted is cytostatic and does not kill the cell (by either the apoptotic or necrotic pathway). In one aspect, the amount eluted is capable of arresting a majority of the smooth muscle cells in the G1/S phase of the cell cycle, without killing the cell.

In certain aspects, the valve prosthesis is capable of eluting a specific amount or percentage of the therapeutic agent(s) incorporated into a coating on the surface of the valve prosthesis.

Coating compositions suitable for forming coatings for the devices of the present invention can comprise one or more therapeutic agents and/or one or more polymeric materials. Optionally, the coating compositions can comprise one, two, three, four, five or more polymeric materials and the polymeric materials can comprise one, two, three, four, five or more therapeutic agents.

The coating composition can comprises a polymeric material incorporating a therapeutic agent. Optionally, the therapeutic agent is paclitaxel. The polymeric material incorporates the paclitaxel or other therapeutic agent by intermixing with the paclitaxel or therapeutic agent, e.g., the polymeric material surrounds at least some of the paclitaxel or therapeutic agent. Optionally, the coating can comprise one or more additional therapeutic agents. In one aspect, the coating comprises a first polymeric material comprising a first therapeutic agent and a second polymeric material comprising a second therapeutic agent. The first and second therapeutic agents can both be the same, e.g., paclitaxel. The first and second therapeutic agents can also differ, e.g., paclitaxel and rapamycin.

To prepare the coating compositions, the constituents, e.g., polymer, paclitaxel, and optionally an additional therapeutic agent, can be suspended and/or dissolved in a solvent. Preferably, the solvent does not alter or adversely impact the therapeutic properties of the therapeutic agent(s) employed. For example, useful solvents for paclitaxel include; polyethoxylated castor oil such as Cremophor EL solution. Inclusion of both the polymeric material and paclitaxel in the coating composition forms a coating wherein the polymeric material incorporates the paclitaxel.

In some aspects, the coating composition comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the polymeric material. In some aspects, the coating composition comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of a (first) therapeutic agent, which is optionally paclitaxel.

In some aspects, the coating composition comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the additional (second, third, fourth, or fifth) therapeutic agent(s).

In a specific embodiment, the coating composition comprises about 0.001 μg, about 0.01 μg, about 0.1 μg, about 1 μg, 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent.

In another aspect, the coating composition comprises about 0.001 μg, about 0.01 μg, about 0.1 μg, about 0.5 μg, about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg or more of the therapeutic agent per mm² of the surface area of the surface of the valve prosthesis.

In certain aspects, the coating composition is capable of releasing a cytostatic amount of a therapeutic agent that is effective of freezing a cell in the G1/S phase.

The coating composition can release about 0.1%, about 1%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95% or more of the paclitaxel incorporated in the polymeric material over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Optionally, the coating composition is capable of releasing about 0.1% to about 35% of an amount of paclitaxel incorporated in the polymeric material over about 1 week to about 8 weeks. Optionally, the coating composition is capable of releasing about 1% to about 15% of an amount of the paclitaxel incorporated in the polymeric material over about 4 weeks.

In another aspect, the coating composition releases about 0.001 μg, about 0.01 μg, about 0.1 μg, about 1 μg, 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Optionally, the coating composition is capable of releasing; about 50 μg to about 200 μg of paclitaxel incorporated in the polymeric material over about 1 week to about 8 weeks.

In yet another aspect, the coating composition releases about 0.001 μg, about 0.01 μg, about 0.1 μg, about 0.5 μg, about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg or more of the therapeutic agent per mm² of the surface area of the surface of the valve prosthesis over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about years, etc. Optionally, the coating composition is capable of releasing about 0.01 μg to about 0.1 μg of paclitaxel incorporated in the polymeric material over about 1 week to about 8 weeks. In certain other aspects, the coating composition is capable of continuously releasing therapeutic agent over a period of time and thereby exposing the cells to a concentration of therapeutic agent that is effective of freezing the cell in the G1/S phase.

Optionally, the concentration of therapeutic agent that a cells is exposed to is about 0.0001 ng/ml, about 0.001 ng/ml, about 0.01 ng/ml, about 0.1 ng/ml, about 1.0 ng/ml, about 10 ng/ml, about 20 ng/ml, about 30 ng/ml, about 40 ng/ml, about 50 ng/ml, about 100 ng/ml, about 200 ng/ml, about 300 ng/ml, about 400 ng/ml, about 500 ng/ml, about 600 ng/ml, about 700 ng/ml, about 800 ng/ml, about 900 ng/ml, about 1,000 ng/ml, about 2,000 ng/ml, about 3,000 ng/ml, about 4,000 ng/ml, about 5,000 ng/ml, about 10,000 ng/ml or more of the one or more therapeutic agents. Optionally, the concentration of therapeutic agent that a cell is exposed to is about 0.001 ng/ml to 10,000 ng/ml of paclitaxel. Optionally, the concentration of therapeutic agent that a cell is exposed to is about 0.01 ng/ml to 1,000 ng/ml of paclitaxel.

A polymeric material suitable for use in the preparation of the coatings of the present invention should be a material that is biocompatible and avoids irritation to body tissue. The polymeric material can be biostable or bioabsorbable. Biostable polymeric materials can be selected from the following: polyurethanes, silicones (e.g., polysiloxanes and substituted polysiloxanes), and polyesters.

Also useful as a polymeric material are styrene-isobutylene copolymers. Other polymers which can be used include ones that can be dissolved and cured or polymerized on the valve prosthesis or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as poly(lactide-co glycolide) (PLGA), polyvinyl alcohol (PVA), poly(L-lactide) (PLEA), polyanhydrides, polyphosphazenes, polycaprolactone (PCL), polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid (PLA), polyglycolic acid (PGA), polyethylene oxide (PEO), polylactic acid-polyethylene oxide copolymers, EPDM (etylene-propylene-diene) rubbers, fluorosilicones, polyethylene glycol (PEG), polyalkylene glycol (PAG), polysaccharides, phospholipids, and combinations of thereof.

In certain aspects, the polymeric material is hydrophilic (e.g., PVA, PLLA, PLGA, PEG, and PAG). In other aspects, the polymeric material is hydrophobic (e.g., PLA, PGA, polyanhydrides, polyphosphazenes and PCL).

For valve prosthesis which undergo mechanical challenges, e.g. expansion and contraction or changes in the beta value or passage shape as described above, the polymeric materials can be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

In some aspects, the polymeric materials are biodegradable. Biodegradable polymeric materials can degrade as a result of hydrolysis of the polymer chains into biologically acceptable, and progressively smaller compounds. Optionally, a polymeric material comprises polylactides, polyglycolides, or their co-polymers.

The polymeric materials can also degrade through bulk hydrolysis, in which the polymer degrades in a fairly uniform manner throughout the matrix. For some novel degradable polymers, most notably the polyanhydrides and polyorthoesters, the degradation occurs only at the surface of the polymer, resulting in a release rate that is proportional to the surface area of the therapeutic agents and/or polymer/therapeutic agent mixtures.

Hydrophilic polymeric materials such as PLGA will erode in a bulk fashion. Various commercially available PLGA may be used in the preparation of the coating compositions.

One skilled in the art will appreciate that the described valves, devices, methods, compositions and agents, can be used to perform a cardiac surgery method for replacing a cardiac valve in a subject comprising removing a cardiac valve of the subject and replacing the removed cardiac valve with a cardiac valve prosthesis. Optionally, the removed cardiac valve of the subject is selected from the group comprising a pulmonary, aortic, mitral, and tricuspid valve.

Throughout this application, various publications are referenced. The disclosures of these publications in their entireties are hereby incorporated by reference into this application in order to more fully describe the compounds, compositions, devices and methods described herein.

The preceding description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. The corresponding structures, materials, acts, and equivalents of all means or step plus function elements in the claims below are intended to include any structure, material, or acts for performing the functions in combination with other claimed elements as specifically claimed.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification. The blocks in the flow charts described above can be executed in the order shown, out of the order shown, or substantially in parallel.

Accordingly, those who work in the art will recognize that many modifications and adaptations to the present invention are possible and can even be desirable in certain circumstances and are a part of the present invention. Other embodiments of the invention will be apparent to those skilled in the art from consideration of the specification and

practice of the invention disclosed herein. Thus, the preceding description is provided as illustrative of the principles of the present invention and not in limitation thereof. It is intended that the specification and examples be considered as exemplary only, with a true scope and spirit of the invention being indicated by the following claims.

What is claimed is:

1. A cardiac valve prosthesis, comprising:
an annulus body defining a passage therethough having a first open end and a second open end, wherein at least a portion of an inner surface of the passage tapers inwardly toward a longitudinal axis of the annulus body as the passage extends from the first open end to the second open end, wherein the diameter of the first open end is greater than the diameter of the second open end, and wherein the valve prosthesis is configured to replace a deficient native valve.

2. The cardiac valve of claim 1, wherein the annulus body further comprises a back face that surrounds at least a portion of the second open end.

3. The cardiac valve of claim 2, wherein the annulus body further comprises a first conduit that is in fluid communication with the back face and the inner surface of the passage.

4. The cardiac valve of claim 3, wherein the first conduit is configured to direct fluid flowing into the passage at a flow angle of 90 degrees or less with respect to the longitudinal axis of the annulus body and in a direction generally toward the second open end.

5. The cardiac valve of claim 4, wherein the flow angle is between about 90 degress and about 30 degrees.

6. The cardiac valve of claim 3, wherein the annulus body further comprises a second conduit that is in fluid communication with the back face and a circumferential outer surface of the annulus body.

7. The cardiac valve of claim 6, wherein the second conduit is in fluid communication with the first conduit.

8. The cardiac valve of claim 1, wherein the valve prosthesis is sized to replace a human pulmonic valve.

9. The cardiac valve of claim 1, wherein the valve prosthesis is sized to replace a human aortic valve.

10. The cardiac valve of claim 1, wherein the valve prosthesis is sized to replace a human atrioventricular valve.

11. The cardiac valve of claim 1, wherein the valve prosthesis is configured such that the passage remains substantially patent during systole and diastole.

12. The cardiac valve of claim 11, wherein at least a portion of the annulus body is sufficiently resilient to allow in situ expansion and contraction throughout the cardiac cycle.

13. The cardiac valve of claim 1, wherein the passage comprises an inwardly tapering portion and a second portion, the inwardly tapering portion extending a predetermined distance from the first open end and is connected to the second portion, and wherein the second portion extends to the second open end.

14. The cardiac valve of claim 13, wherein the inwardly tapering portion of the passage has a substantially elliptical curvature is cross-section.

15. The cardiac valve of claim 13, wherein the inwardly tapering portion of the passage has a curvature in cross-section that is selected from the group consisting of a circle, a cubic polynomial, and spline based.

16. The cardiac valve of claim 13, wherein the second portion of the passage extends substantially parallel to the longitudinal axis of the annulus body.

17. The cardiac valve of claim 13, wherein the second portion of the passage is outwardly tapering as it extends to the second open end of the annulus body.

18. The cardiac valve of claim 1, wherein the ratio of the diameter of the first open end to the diameter of the second open end is between about 0.4 and about 0.8.

19. The cardiac valve of claim 18, wherein the ratio is between about 0.5 and about 0.7.

20. The cardiac valve of claim 1, wherein the annulus body is resilient.

21. The cardiac valve of claim 20, wherein at least a portion of the passage is configured to narrow during diastole.

22. The cardiac valve of claim 20, wherein the annulus body is configured to the collapsible for transluminal delivery.

23. The cardiac valve of claim 22, wherein the annulus body is configured to be expandable to contact the anatomical annulus of the native valve when the valve prosthesis is positioned in situ.

24. The cardiac valve of claim 1, wherein the annulus body further comprises an anchor for engaging the lumen wall for preventing substantially migration of the valve prosethesis after placement in the desired anatomical position.

25. The cardiac valve of claim 24, wherein the anchor comprises at least one circumferentially extending ring member extending outwardly from an outer surface of the annulus body, whereby the ring member is configured for suturing.

26. The cardiac valve of claim 1, wherein the annulus body comprises natural tissue.

27. The cardiac valve of claims 1 or 26, wherein the annulus body comprises synthetic material.

28. The cardiac valve of claim 1, wherein the annulus body is self-expanding.

29. The cardiac valve of claim 24, wherein the annulus body comprises wire.

30. The cardiac valve of claim 2, wherein the back face of the annulus body defines a circumferentially extending trough that has a substantially hook-shaped cross-sectional shape.

31. A method of replacing a deficient native valve, comprising the steps of:
providing a cardiac valve prosthesis comprising an annulus body defining a passage therethough having a first open end and a second open end, the passage having an inner surface, wherein at least a portion of the inner surface of the passage tapers inwardly toward a longitudinal axis of the annulus body as the passage extends inwardly from the first open end to the second open end, and wherein the diameter of the first open end is greater than the diameter of the second open end;
collapsing the annulus body to fit within a distally positioned sheath on a catheter;
advancing the catheter to the deficient native valve;
deploying the annulus body of the valve prosthesis; and
withdrawing the catheter, leaving the valve prosthesis to function in place of the deficient native valve.

32. The method of claim 26, further comprising the step of excising the native valve.
